(19) United States
(12) Patent Application Publication
(10) Pub. No.: US 2004/0151700 A1
(43) Pub. Date: Aug. 5, 2004

(54) Ex-vivo rescue of transplantable hematopoietic stem cells following myeloablative injury

(76) Inventors: David M. Harlan, Potomac, MD (US); Thomas Davis, Newtown, PA (US); John Chute, Bethesda, MD (US)

Correspondence Address: NAVAL MEDICAL RESEARCH CENTER, ATTN: (CODE 00L), 503 ROBERT GRANT AVENUE, SILVER SPRING, MD, 20910-7500, US

(21) Appl. No.: 10/283,123

(22) Filed: Oct. 30, 2002

Related U.S. Application Data
(60) Provisional application No. 60/330,754, filed on Oct. 30, 2001.

(51) Int. Cl. A61K 45/00; C12N 5/08

(52) U.S. Cl. 424/93.7; 424/85.1; 424/85.2; 435/372

(57) ABSTRACT

An ex-vivo method of treating myeloablation of hematopoietic stem and progenitor cells, particularly myeloablation due to ionizing radiation is disclosed. An ex-vivo method of restoring a depleted population of rapidly proliferating hematopoietic stem cells is also disclosed. The methods comprise co-culturing resistant hematopoietic stem cells in a culture medium comprising a monolayer of endothelial cells and various cytokines. Bone marrow stem cells harvested from animals exposed to 1050 cGy were incapable of providing hematopoietic recovery in secondary irradiated recipients. Bone marrow stem cells harvested from animals exposed to 1050 cGy and then co-cultured×10 days with endothelial cell monolayers showed complete recovery of hematopoietic repopulating capacity which was equivalent to normal BM stem cells.

### CROSS-REFERENCE TO RELATED APPLICATIONS

This application claims priority to U.S. Provisional Application 60/330,754, filed Oct. 30, 2001, entitled EX VIVO RESCUE OF TRANSPLANTABLE HEMATOPOIETIC STEM CELLS FOLLOWING HIGH DOSE RADIATION INJURY. This application hereby incorporates by reference and in its entirety, U.S. patent application Ser. No. 09/452,855, filed Dec. 3, 1999, entitled HUMAN BRAIN ENDOTHELIAL CELLS AND GROWTH MEDIUM AND METHOD FOR EXPANSION OF PRIMITIVE CD34+CD38− BONE MARROW STEM CELLS, by Chute et al. as well as references 1 through 36 cited and listed in this application.

### FIELD OF THE INVENTION

The invention relates to the ex-vivo rescue of hematopoietic stem and progenitor cells following myeloablative injury. Specifically, the invention relates to the rescue of hematopoietic stem cells with repopulating capacity from animals exposed to high dose radiation.

### BACKGROUND OF THE INVENTION

Myeloablative injury can be the result of disease, viral infections (e.g. HIV), genetic disorders, drugs, toxins, and radiation as well as many therapeutic treatments, such as high-dose chemotherapy and conventional-dose oncology therapy. Damage to the bone marrow precursors results in pancytopenia (reduction in all cell lines produced in the bone marrow). The clinical manifestations include thrombocytopenia with subsequent increased risk of bleeding, anemia, and leukopenia with increased risk of infection. Transfusions of red blood cells and/or platelets may be required. Patients suffering from the resulting leukopenia and neutropenia are at increased risk from infection as the diminished number of neutrophils circulating in the blood substantially impairs the ability of the patient to fight infection. Treatment of various cancers increasingly involves cytoreductive therapy, including high doses of chemotherapy or radiation therapy that are also myeloblative or severely myelosuppressive. These therapies decrease a patient's white blood cell counts, suppress bone marrow hematopoietic activity, and increase their risk of infection and/or hemorrhage. As a result, patients who undergo cytoreductive therapy must also receive therapy to reconstitute bone marrow function (hematopoiesis).

Several methods are directed towards restoring the patients immune system after therapy. Hematopoietic growth factors are administered after therapy to stimulate remaining stem cells to proliferate and differentiate into mature infection fighting cells. Although hematopoietic growth factors can shorten the total period of neutropenia, there remains a critical 10-15 day period immediately following therapy when the patient is severely neutropenic and thus infection prone. Another approach to the management of the problems that result from prolonged bone marrow suppression includes the reinfusion of a patient's own previously harvested peripheral blood progenitor cells (PBPC), known as autologous stem cell transplantation or autologous stem cell rescue. In such procedures, patients undergo successive treatments with cell mobilization agents to cause mobilization of hematopoietic progenitor cells from the bone marrow to the peripheral circulation for harvesting.

Growth factors used for mobilization include interleukin-3 (IL-3), granulocyte colony stimulating factor (G-CSF), granulocyte-macrophage colony stimulating factor (GM-CSF), stem cell factor (SCF) and a recombinant fusion protein having the active moieties of both IL-3 and GM-CSF (Brandt, S J, et al., N Eng J Med 318:169, 1988; Crawford, J et al. N Eng J Med 325:164, 1991; Neidhart, J, et al., J Clin Oncol 7:1685, 1989). After harvesting, the patient is given high dose chemotherapy or radiotherapy and the bone marrow function is reconstituted by infusion of the cells harvested earlier.

The effects of ionizing radiation on the hematopoietic stem cell compartment have been extensively studied. In murine models, the radiation sensitivity of hematopoietic progenitor cells has been measured based upon these cells' marrow repopulating ability (MRA) post-radiation exposure as well as colony forming unit-spleen (CFU-S) assays. In these studies, it has been demonstrated that a dose of 500 Centigray (cGy) of ionizing radiation eliminates 99% of the competent hematopoietic stem cells based upon their ability to repopulate a lethally irradiated secondary recipient.

Despite these data, it has been postulated that a small subset of hematopoietic stem cells may be radio-resistant. This hypothesis has been based upon observations in mice that a small fraction of spleen colony forming unit cells (CFU-S) were capable of surviving doses of ionizing radiation up to 600 cGy. Anecdotal observation of hematopoietic recovery in humans following exposure to nuclear fallout has also suggested this possibility. Low dose radiation induces cellular apoptosis via activation of Fas ligand mediated pathways whereas higher radiation exposures induce double-stranded DNA damage which is most rapidly lethal in dividing cells. We and others have shown that the most primitive bone marrow stem cells are highly quiescent in the steady state. It is therefore plausible that the most primitive hematopoietic stem cells residing in G₀ might possess the capacity to repair radiation-induced DNA damage.

Ionizing radiation abrogates the hematopoietic function of the bone marrow via its effects on both hematopoietic stem cells and the marrow stromal microenvironment. Stromal cells are both mesenchymal and hematopoietic in origin, and include osteoblasts, fibroblasts, adipocytes, myocytes, endothelial cells, dendritic cells and macrophages. Direct toxic effects of irradiation on stromal cell lines have been demonstrated and irradiated stromal cells release high levels of nitric oxide which would likely contribute to the demise of neighboring hematopoietic stem cells in vivo. Therefore, extraction of irradiated hematopoietic stem cells from the injured marrow microenvironment could offer theoretical benefits toward increasing the survival of these cells.

### SUMMARY OF THE INVENTION

Various cytokines, including flt-3 ligand, IL-1, TNF-alpha, and SCF, have at least partial radioprotective effects in mice when administered prior to or at the time of radiation exposure. However, the in vitro rescue of hematopoietic stem cells harvested from animals after high dose radiation injury has not been previously demonstrated. We have developed in vitro systems using primary brain endothelial cell lines, particularly porcine brain microvascular endothelial cells (PMVEC) and human brain endothelial cells (HUBEC), which support the maintenance and expansion of steady state bone marrow stem cells capable of in vivo repopulation. Increasing evidence also has verified the critical role of endothelial cell precursors in the development of the hematopoietic system during embryogenesis, as well as the possibility of a common endothelial/hematopoietic precursor cell, the hemangioblast. We hypothesized that a fraction of hematopoietic stem cells exposed to high dose irradiation might be recoverable if these cells were co-cultured ex-vivo with endothelial monolayers, specifically, PMVEC or HUBEC cells. We tested this by comparing the in vivo repopulating capacity of the irradiated/co-cultured cells versus non-cultured hematopoietic stem cells which were exposed to the identical dose of radiation. Our results indicate that ex-vivo co-culture with endothelial cell monolayers, particularly, PMVEC or HUBEC monolayers completely rescues the in vivo repopulating capacity of bone marrow stem cells which is otherwise destroyed following high dose ionizing radiation. A rare fraction of bone marrow stem cells appears to be radioresistant to high dose radiation, but this radioresistance may depend upon repair signaling from endothelial cells.

Accordingly, there is a need for a method of treating the myeloablation of hematopoietic stem and progenitor cells in a subject which includes isolating hematopoietic stem and progenitor cells from the subject and expanding the isolated stem and progenitor cells in a co-culture medium including endothelial cells. The method also includes harvesting expanded hematopoietic stem cells from the co-culture medium and administering a therapeutic dose of the harvested expanded stem and progenitor cells to the subject.

There is also a need for a method of restoring a depleted population of rapidly proliferating hematopoietic stem cells which includes isolating hematopoietic stem and progenitor cells from a donor and expanding the isolated stem and progenitor cells in a co-culture medium which includes endothelial cells. The method further includes harvesting expanded hematopoietic stem and progenitor cells from the co-culture medium and administering a therapeutic dose of the harvested and expanded hematopoietic stem cells to a subject.

There is also a need for an ex-vivo co-culture system that supports the recovery of CD34+ bone marrow hematopoietic stem or progenitor cells following myeloablative injury that is not associated with the development of engraftment defects in the co-cultured cells, the differentation of the stem cell population, and alterations in adhesion molecules/homing receptors on the expanded hematopoietic cells.

### DESCRIPTIONS OF THE DRAWINGS

FIG. 1. Schema of experimental transplantation procedures. C57B16 donor mice (Ly 5.1) were irradiated with 1050 cGy (split dose) and their bone marrow (BM) was subsequently harvested. Purified BM mononuclear cells (MNC) were obtained via Ficoll-Hypaque centrifugation. A group of Ly 5.2 mice (Group 1) were then irradiated with 1050 cGy and then transplanted via tail vein injection with 2×10⁶ irradiated BM MNC per mouse. A portion of irradiated BM MNC from donor Ly 5.1 mice were placed in culture×10 days with PMVEC monolayers supplemented with GMCSF+IL-3+IL-6+SCF+Flt-3 ligand. After 10 days, the non-adherent hematopoietic cells were collected from these cultures and injected via tail vein infusion into irradiated Ly 5.2 mice (Group 2) at a dose equal to the dose given to Group 1. As a positive control (Group 3), Ly 5.2 mice were irradiated with 1050 cGy and then transplanted with 2×10⁶ normal Ly 5.1 donor BM MNC. All animals were followed for 8 weeks post-transplantation.

FIG. 2. PMVEC culture supports the recovery of irradiated hematopoietic progenitor cells and colony forming cells. FIG. 2(A) shows hematopoietic cell counts during culture were measured over time at days 0, 3, 7, and 10 following exposure of donor mice to 1050 cGy. The cell count curves are identified at right as either PMVEC (open squares; normal BM MNC cultured with PMVEC monolayers supplemented with GMCSF/IL-3/IL-6/SCF/Flt-3 ligand), LIQUID (filled diamond; normal BM MNC cultured with stroma-free liquid culture plus identical cytokines), 1050 cGy+PMVEC (open circles; or 1050 cGy+LIQUID (filled triangles). Each data point represents the mean number of viable cells counted in culture at each time point. Experiments were performed in triplicates and the error bars represent the standard errors of the mean. FIG. 2(B) shows log scale of bar graphs showing the mean number of CFU-GM, BFU-E, CFU-Mix, and CFU-Total measured in 14 day methylcellulose cultures (n=3 experiments) from each of 3 groups: Normal (non-irradiated) BM MNC (white bars), Irradiated/PMVEC cultured cells (black bars), and non-cultured irradiated cells (gray bars). Error bars represent the standard errors of the mean.

FIG. 3. Light microscopic view of irradiated hematopoietic cells during PMVEC culture vs. stroma-free liquid culture. FIG. 3(A) shows a small colony of hematopietic cells adherent to PMVEC monolayers, seen at 72 hours post-radiation. FIG. 3(B) shows an image of cells from the same donor at 72 hours post-radiation in stroma-free liquid culture shows few viable cells. At day 7 post-radiation, an expanding colony of hematopoietic cells can be visualized in FIG. 3(C) on PMVEC monolayers, however, cell debris and crenated hematopoietic cells predominate within stroma-free liquid cultures as shown in FIG. 3(D). At day 10 post-radiation, sheets of hematopoietic cells are evident within PMVEC cultures as shown in FIG. 3(E). FIG. 3(F) shows Wrights Geimsa stain of hematopoietic progenitor cells adherent to PMVEC monolayers at day 10 post-radiation. The hematopoietic cells are monomorphic with high nuclear:cytoplasmic ratios consistent with immature progenitors/stem cells. Endothelial cells can be seen in the background of the hematopoietic cells.

FIG. 4. Transplantation of irradiated/PMVEC-cultured cells increases the survival of irradiated recipient mice. Kaplan-Meier curve demonstrating survival of 3 groups of mice: 1) Ly 5.2 recipient mice were irradiated with 1050 cGy and then transplanted with either 2×10⁶ irradiated/PMVEC-cultured cells (solid line: 1050 cGy+1050 cGy/PMVEC; n=11), 2) 2×10⁶ irradiated/non-cultured cells (hatched line: 1050 cGy; n=10), or 3) 2×10⁶ normal (non-irradiated) BM MNC (dotted line: 1050 cGy+normal BM MNC; n=10). All mice in each group were followed for 8 weeks post-transplantation. The Y axis shows the percent of animals surviving at each time point.

FIG. 5. Representative engraftment of irradiated/PMVEC cultured donor Ly 5.1 cells in the bone marrow of Ly 5.2 recipient mice at 8 weeks post-transplantation. FIG. 5(A) shows the staining of a normal female (Ly 5.2) mouse BM cells with the Ly 5.1 antibody is shown. The isotype control is shown at left. FIG. 5(B) shows the expression of Ly 5.1 within the bone marrow of a female Ly 5.2 mouse at 8 weeks following 1050 cGy irradiation and transplantation with 1050 cGy irradiated/PMVEC cultured donor Ly 5.1 cells. The isotype control is shown at left. FIG. 5(C) shows the initial flow cytometry gating of bone marrow cells from a representative Ly 5.2 mouse transplanted with PMVEC-cultured cells (Ly 5.1) is shown in the top left panel demonstrating the exclusion of non-viable cells. The expression of B220 (y axis) and Ly 5.1 (x axis) on bone marrow cells from Ly 5.2 recipient transplanted with PMVEC cultured Ly 5.1 cells is shown at top right. The expression of CD3 (y axis) and Ly 5.1 (x axis) from this recipient is shown in the bottom left panel. The expression of MAC-1 (y axis) and Ly 5.1 (x axis) is shown in the bottom right figure. Percentages of cells expressing each phenotype are shown within each quadrant.

### DETAILED DESCRIPTION OF THE INVENTION

The present inventive subject matter involves an ex-vivo method of treating myeloablation of hematopoietic stem and progenitor cells from a myeloablated subject which includes the steps of isolating the hematopoietic stem and progenitor cells from a subject and expanding the isolated stem and progenitor cells in a co-culture medium including endothelial cells. The expanded stem and progenitor cells are then harvested from the co-culture medium. A therapeutic dose of the harvested and expanded stem and progenitor cells is then administered back to the subject.

The inventive subject matter also includes an ex-vivo method of restoring a depleted population of rapidly proliferating hematopoietic stem and progenitor cells which comprises the steps of isolating hematopoietic stem and progenitor cells from a donor and expanding the isolated hematopoietic stem and progenitor cells in a co-culture medium including endothelial cells. The expanded hematopoietic stem and progenitor cells from the co-culture medium are then harvested and a therapeutic dose of the harvested expanded stem cells is administered to a subject. See the Conclusions following Example 3.

Myeloablative injury can occur for a variety of reasons such as disease, genetic disorders, drugs, toxins, and ionizing radiation as well as many therapeutic treatments, such as high dose chemotherapy and conventional oncology therapy, resulting in the need for bone marrow transplantion.

The dose limiting side effects of chemotherapy and radiation therapy are their deleterious effects on hematopoietic cells through destruction of the bone marrow stem cells which are the precursor cells for all hematopoietic cells. This damage to the marrow results in myelosuppression or myeloablation, rendering patients susceptible to opportunistic infections for a prolonged period of time. Bone marrow transplantation involves the infusion of early bone marrow progenitor cells that have the ability to re-establish the patients' hematopoietic system, including the immune system. Transplantation decreases the time normally required for the restoration of the immune system after chemotherapy or radiation therapy and, thus, the time of risk for opportunistic infections.

The pluripotent hematopoietic stem cell can be defined functionally as well as phenotypically. Functionally, stem cells are those hematopoietic cells having the capability for prolonged self-renewal as well as the ability to differentiate into all the lymphohematopoietic cell lineages. Thus, pluripotent hematopoietic stem cells, when localized to the appropriate microenvironment, can completely and durably reconstitute the hematopoietic and lymphoid compartments. Multilineage stem and progenitor cells can also be identified phenotypically by cell surface markers. A number of phenotypic markers, singly and in combination, have been described to identify the pluripotent hematopoietic stem cell. Primitive human hematopoietic stem cells have been characterized as small cells which are CD34⁺, 38⁻, HLA⁻DR⁻, Thy1^{+/-}, CD15⁻, Lin⁻, c-kit⁺, 4-hydroperoxycyclophosphamide-resistant and rhodamine 123 dull. Equivalent primitive murine stem cells have been characterized as Lin⁻, Sca⁺, and Thy1.1⁺. Preferably, the human hematopoietic stem cells of the present methods are CD34⁺CD38⁻. Differentiated hematopoietic stem cells are CD34⁺CD38⁺.

In particular, the ex-vivo rescue of hematopoietic stem cells following high dose radiation injury has not been previously demonstrated. Previous studies have indicated that bone marrow stem cells are highly sensitive to ionizing radiation [1-3]. In mice, the radiosensitivity (Do) of the most primitive assayable hematopoietic progenitor cells has been estimated to range from 0.71 to 1.38 Gy with 99% of long term repopulating cells being eliminated following exposure to 500 cGy [1, 26]. Therefore, it has been considered implausible that a clinically relevant number of bone marrow repopulating cells might be recoverable following high dose radiation exposure. Despite this, we have found that bone marrow stem cells harvested from animals exposed to high dose radiation can be rescued via ex-vivo culture with endothelial monolayers supplemented with GMCSF/IL-3/IL-6/SCF/Flt-3 ligand.

We define the isolating step of the present inventive methods as the act of collecting cells from a bone marrow aspirate and using various physical means, known to those of skill in the art, to enrich for CD34+ mononuclear cells.

We define the harvesting step as the act of washing non-adherent cells off the PMVEC or HUBEC co-culture system. This cell population is ultimately administered to the subject.

It will be appreciated by those of skill in the art, that the present inventive methods contemplates that the donor and subject may be autologous or heterologous. It will be further appreciated that while the subject is myeloablated, that the donor may or may not be myeloablated.

### Use of the Methods

The method of treating myeloablation of hematopoietic stem and progenitor cells of the present inventive subject matter involves isolating hematopoietic stem and progenitor cells from the bone marrow, peripheral blood or umbilical cord using methods and materials known in the art, described in the bone marrow stem cell isolating procedure of U.S. Pat. No. 5,599,703, col. 11, lines 27-41, which is hereby incorporated by reference. As the present method is useful for amplifying/expanding stem cells from various species that have had exposure to myeloablative agents, the stem and progenitor cells can be isolated from, for example, humans, non-human primates or mice. The stem and progenitor cells utilized in the present method are preferably substantially enriched, that is depleted of mature lymphoid and myeloid cells. Preferably, the hematopoietic stem and progenitor cells are enriched at least 85%, more preferably at least 95%, and most preferably at least 99%. Several methods by which CD34+ stem and progenitor cells can be isolated and enriched to high degrees of purity using positive immunoselection have been described by Berenson et al (Journal of Immunological Methods, 91: 11-19, 1986), Thomas et al (Prog Clin Biol Res 377:537-44, 1992) and Okama et al (Prog Clin Biol Res 377:487-502, 1992)

In the present method of treatment and culture system, the enriched hematopoietic stem and progenitor cells are placed in direct contact with endothelial cells supplemented with GMCSF/IL-3/IL-6/SCF/Flt-3 ligand. Preferred endothelial cells are brain microvascular endothelial cells, more particular porcine brain microvascular endothelial cells (PMVEC). Examples of other endothelial cells suitable for use in the inventive subject matter include, but are not limited to, brain endothelial cells, human brain endothelial cells (HUBEC), human endothelial cells, microvascular endothelial cells, porcine endothelial cells and various types of immortalized endothelial cells. The method of preparation of the endothelial cell culture and culture conditions is as described in U.S. Pat. No. 5,599,703, col. 14 lines 30-67-col.15, lines 1-13, and is hereby incorporated by reference.

It is important that the hematopoietic stem and progenitor cells be in contact with the endothelial cells to maximize amplification/expansion. For example, the hematopoietic stem and progenitor cells can be seeded onto a 70-100% semi-confluent monolayer of PMVECs. Amplification/expansion of primitive hematopoietic stem and progenitor cells in vitro increases significantly within 7-14 days when the stem and progenitor cells are directly cultured on endothelial cells and supplemented with at least one cytokine, preferably GMCSF/IL-3/IL-6/SCF/Flt-3 ligand.

Preferably, the hematopoietic stem and progenitor cells are isolated from the subject within 24 hours of the myeloablative injury as the toxic effects of ionizing irradiation, including release of nitric oxide, can contribute to the death of neighboring hematopoietic stem cells in vivo. The culture medium of the present methods are preferably maintained at a pH of about 7.2 to about 7.5 while the isolated hematopoietic stem cells are being expanded. The pH of the culture medium is maintained by replacing a portion of the culture medium.

### Dosage and Routes of Administration

Once the hematopoietic stem and progenitor cells have been expanded and harvested from the co-culture medium, a therapeutic dose is administered to a subject. The method of determining an appropriate therapeutic dose is known to those of skill in the art; however, the inventors have determined that, preferably, a therapeutic dose is 1 to about 2 million cells/kg of the subject's mass. Preferably, the therapeutic dose is administered intravenously.

### Observations

In the present inventive method of treating, irradiated bone marrow cells appeared to completely re-acquire their in vivo repopulating capacity during the 10 day co-culture period. In contrast, irradiated bone marrow stem cells which were not co-cultured showed no in vivo repopulating capacity. In addition, stroma-free liquid culture supplemented with GMCSF/IL-3/IL-6/SCF/Flt-3 ligand failed to support the recovery of hematopoietic stem cell numbers or colony forming cells, thereby highlighting the importance of the endothelial cell monolayers in the recovery process following radiation injury.

The mechanism through which PMVEC culture supports the recovery of irradiated hematopoietic stem cells is not clear. It has been previously shown in animal models that administration of TNF, IL-1, flt-3 ligand, or SCF prior to or at the time of radiation exposure can protect the hematopioetic compartment against terminal injury [16-18]. TNF has been shown to induce cells into Go, whereas IL-1, SCF, and Flt-3 ligand appear to induce cells into late S phase which is a less radiosensitive stage of cell cycle [16-18]. We have previously shown that PMVEC culture induces a high level of cell cycling in the most primitive stem cell populations [11]. Therefore, it is unlikely that PMVEC culture induced a significant percentage of BM stem cells into Go as a mechanism of recovery post radiation injury. We have not studied whether PMVEC elaborate anti-oxidants (e.g. lipoic acid, superoxide dismutase) which might promote the recovery of stem cells post radiation injury [27, 28], but it is plausible that novel soluble or membrane-associated factors elaborated by PMVEC contribute to stem cell repair following radiation injury.

In addition to the reparative factors which PMVEC might provide in co-culture to promote hematopoietic stem cell recovery, the removal of stem cells from the irradiated bone marrow microenvironment is also likely to be beneficial to these cells' survival. Ionizing radiation has been shown to decrease bone marrow stromal cells' hematopoietic capacity, damage their adherence capacity, and induce the release of toxic molecules [15, 29, 30]. Therefore, removal of hematopoietic stem cells from the irradiated marrow microenvironment and placement in co-culture with PMVEC may have increased the fraction of stem cells which could be recovered following radiation damage. It is possible that the ex-vivo culture of irradiated bone marrow stem cells with a “healthy”, non-damaged stromal cell line might also have supported the recovery of a percentage of repopulating cells. In this study, we elected to evaluate the restorative capacity of PMVEC first since we have found this cell line to be highly potent with regard to hematopoietic progenitor cell expansion as compared to the activity of recently described stromal cell lines [31, 32].

In this study, we have attempted preliminarily to determine whether ex-vivo culture could enrich for a radioresistant hematopoietic cell population. Although the existence of a “radioresistant” stem cell population has been hypothesized (4), the isolation and characterization of this rare population has yet to be achieved. Our first goal was to demonstrate whether a population of bone marrow progenitor cells could be functionally rescued under optimized ex-vivo culture conditions. However, we have not yet identified the specific sub-population of murine bone marrow cells which undergo such recovery following radiation injury.

Since PMVEC co-culture is associated with both the recovery of colony forming capacity and in vivo repopulating capacity within heavily irradiated bone marrow cells, it appears that PMVEC co-culture restores or enhances critical functions within the progenitor cell population during the 10 day co-culture period. Alternatively, since PMVEC co-culture also promotes the recovery of progenitor cell numbers following radiation injury to input levels, PMVEC may also be increasing the frequency of repopulating cells so that engraftment can be observed in the transplanted recipients. In order to further test the importance of cell dose in this radiation injury model, we will be testing whether logarithmically higher doses of irradiated bone marrow MNC (2×10⁷, 2×10⁸) are capable of engrafting in secondary, irradiated recipients. We will also be performing cell sorting experiments on candidate stem cell subsets (Sca-1⁺kit⁺ CD34⁺ CD38⁻, Sca-1⁺Thy1.1^{lo}) [25, 33] in order to determine which sub-population accounts for the engraftment and recovery of function following high dose radiation injury.

The mortality which is observed during the first 28 days following radiation injury is causally associated with the decline in committed, dividing hematopietic cells rather than the effect of damage to the primitive stem cell compartment. Since PMVEC co-culture supported the nearly complete in vitro recovery of CFU-Total within bone marrow cells following high dose radiation injury, these PMVEC co-cultured grafts were likely to contain committed, short-term engrafting cells which would be capable of ameliorating the short-term toxicity of radiation in recipient mice. The significant 30 day survival difference between control irradiated mice and mice which received irradiated/PMVEC cultured cells also supports this conclusion. However, the high percentage of donor-derived cells measured within the bone marrow of mice which received PMVEC-cultured cells 8 weeks post-transplant verifies that the PMVEC-cultured grafts also contained a significant frequency of long term repopulating cells. Therefore, we hypothesize that PMVEC-cultured grafts provided both short-term and long-term engrafting cells and radioprotection, whereas host long-term repopulating cells appeared to have little contribution to the survival of the transplanted recipient mice.

### Surprising Results

Our observation that ex-vivo co-culture supports the recovery of bone marrow stem cells following high dose radiation injury is most surprising in light of previous studies which have demonstrated the ineffectiveness of ex-vivo culture in maintaining normal (non-irradiated) bone marrow stem cells with repopulating capacity [34-36]. Ex vivo culture of bone marrow stem cells has been associated with the development of an engraftment defect in the cultured cells, the differentiation of the stem cell population, and alterations in adhesion molecules/homing receptors on the expanded hematopoietic cells [34-36]. Our current results using the PMVEC line illustrate the potency of primary brain-derived endothelial cells in the ex-vivo maintenance of hematopoietic stem cells. As importantly, these results have clinical implications for the management of myeloablation victims. Heretofore, patients who have suffered myeloablative injury have typically succumbed to the morbidities of bone marrow aplasia (infection, bleeding complications) despite optimized supportive care. Allogeneic stem cell transplantation has been considered the only definitive therapeutic option in such circumstances. Our current model raises the new possibility that rapid ex-vivo culture of autologous BM cells from radiation victims using specialized endothelial monolayers can generate classes of repopulating progenitor cells capable of providing complete in vivo hematopoietic recovery.

Having generally described the invention, the same will be more readily understood through reference to the following examples which are provided by way of illustration and are not intended to be limiting.

### EXAMPLE 1

### Ex-Vivo Cultures of Irradiated Bone Marrow

Porcine microvascular endothelial cell (PMVEC) cultures and stroma-free liquid cultures were initiated and supplemented with GMCSF+IL-3+IL-6+SCF+Flt-3 lig as previously described. PMVECs were plated at cellular concentrations of 1×10⁵ cells/well in gelatin-coated 6-well tissue culture plates (Costar, Cambridge, Mass.) containing 5 mL of M199 supplemented with 10% heat-inactivated FCS (Hyclone, Logan, Utah), 100 mcg/mL L-glutamine, 50 mcg/mL heparin, 30 mcg/mL endothelial cell growth factor supplement (Sigma, St. Louis, Mo.) and 100 mcg/mL penicillin/streptomycin solution. After 48-72 hr, the adherent PMVEC monolayers (70-80% confluent) were washed twice with complete culture medium to remove any non-adherent PMVEC and the culture medium was replaced with 5 mL of complete cell culture medium. 4×10⁵ irradiated (1050 cGy) murine bone marrow mononuclear cells were added to each well. Cultures were treated with 2 ng/mL mu-GM-CSF, 5 ng/mL mu-IL-3, 5 ng/mL mu-IL-6, 120 ng/mL mu-SCF, and 50 ng/mL hu-Flt-3 ligand (R & D Systems, Minneapolis, Minn.) and incubated at 37° C. in humidified 5% CO₂-in-air atmosphere. After 7 days, and additional 5 mL of complete culture medium plus the above cytokines were added to each well. At day 10, the PMVEC monolayers were washed to remove both the adherent and non-adherent hematopoietic cells and the harvested cells were washed, and manual hemacytometer cell counts were performed using trypan blue exclusion dye. The day 0 bone marrow mononuclear cells and the expanded day 10 hematopoietic cells were each stained with MoAb anti-Sca-PE and anti-Thy 1.1 FITC and the expression of these antigens was compared to the isotype IgG PE and IgG FITC controls. Stroma-free liquid cultures were performed using the identical cytokine combination as a control. Day 0, 3, 7, and 10 cell counts were each performed in triplicate.

### EXAMPLE 2

### Colony Forming Assays

Colony forming assays were performed using a modification of the technique previously described [11]. Briefly, 5-50×10² BM cells were seeded into 1 mL of IMDM (Gibco, Grand IsLand, N.Y.), 1% methylcellulose, 30% heat-inactivated FCS, 10 U/mL recombinant human erythropoietin, 2 ng/mL mu-GM-CSF, 10 ng/mL mu-IL-3, and 120 ng/ml mu-SCF (R&D Systems, Minneapolis, Minn.). After 14 days, cultures were evaluated to determine the number of colonies (>50 cells) developed. Aggregates of hemoglobin containing cells were recognized as BFU-E, granulocyte-macrophage colonies as CFU-GM and aggregates of hemoglobin cells containing at least granulocytes and/or macrophages and/or megakaryocytes as CFU-Mix. Morphological verification of selected colonies was determined using Wrights-Giemsa stain. Triplicate assays were set up for each individual data point per experiment.

### EXAMPLE 3

### Transplantation of Irradiated BM cells Into Irradiated Recipients

Six to 8 week old C57BL6 (Ly 5.2) and C57BL6J (Ly 5.1) mice were used as recipient and donor mice, respectively [25]. Donor Ly 5.1 mice were irradiated with a split dose of 1050 cGy (550 cGy and 500 cGy separated by 4 hrs) delivered by a ¹³⁷CS irradiator at a rate of 137 cGy/minute. Two hours subsequently, the animals were sacrificed and their bone marrow was collected by flushing both femurs with cold (4° C.) PBS plus 10% FCS. The collected cells were washed×2 and then the mononuclear cell fraction was isolated using Ficoll-Hypaque separation. These cells were then either placed in co-culture with PMVEC monolayers (starting dose 4×10⁵ cells/well×10 days), stroma-free cultures, or injected via tail vein infusion at a dose of 2×10⁶ cells into irradiated Ly 5.2 recipients. In the PMVEC culture group, the non-adherent cells were collected at day 10, washed×2, and then transplanted into irradiated (1050 cGy split) recipient Ly 5.2 mice at a dose of 2×10⁶ cells per animal. As positive controls, a group of Ly 5.2 animals were irradiated and then transfused with 2×10⁶ non-irradiated bone marrow MNC from Ly 5.1 mice. Engraftment of Ly 5.1 cells in Ly 5.2 mice was measured at week 8 following transplantation when the recipient animals were sacrificed and bone marrow MNC were stained with anti-Ly 5.1 MoAb and compared with the isotype IgG control fluorescence using FACS.

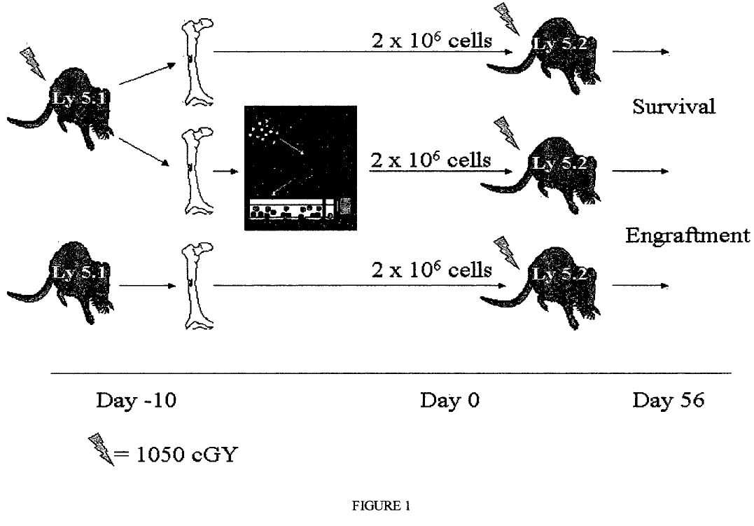

Animals in the experimental and control groups were transplanted and followed for survival following the schema shown in FIG. 1. All animals in each group were injected with 2×10⁶ hematopoietic cells per graft. We irradiated donor (Ly 5.1) and recipient (Ly 5.2) animals with 1050 cGy (550 cGy/500 cGy split dose) because we found this to be the LD_{80/30} for 6 week old C57BL6 mice in our preliminary studies, and this dose was 2-fold higher than the radiation doses which have been previously shown to eliminate the in vivo repopulating capacity of murine hematopoietic stem cells [1]. Of note, neither experimental nor control mice received antibiotics following radiation treatments.

Statistical Analysis

The comparison between the recovery of irradiated hematopoietic progenitor cells during PMVEC culture and stroma-free liquid culture was measured using the student's t test. The Wilcoxon rank sum test was used to compare the CFC capacity of irradiated/PMVEC cultured cells vs. irradiated/stroma-free cultured cells vs. irradiated/uncultured cells. The student's t test was utilized to compare the survival durations of animals transplanted with irradiated BM MNC vs. animals transplanted with irradiated/PMVEC cultured cells vs. animals which received normal BM MNC.

### Results

In Vitro Recovery of Irradiated BM Progenitor Cells

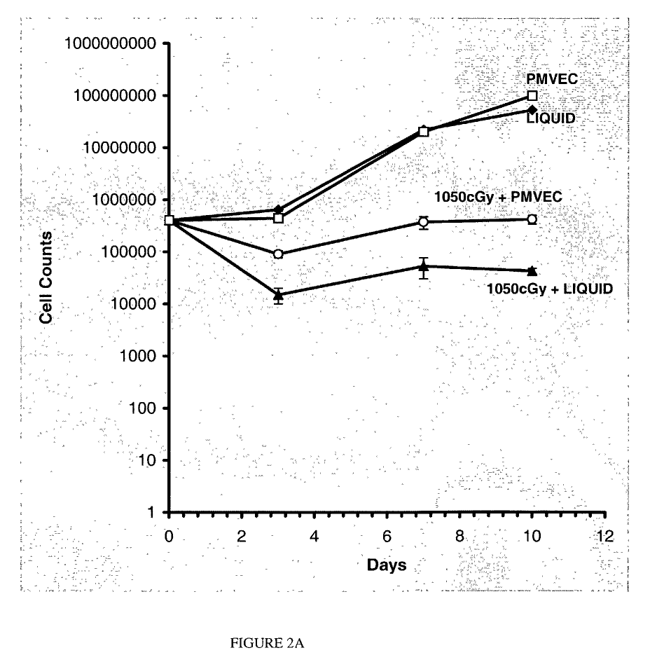

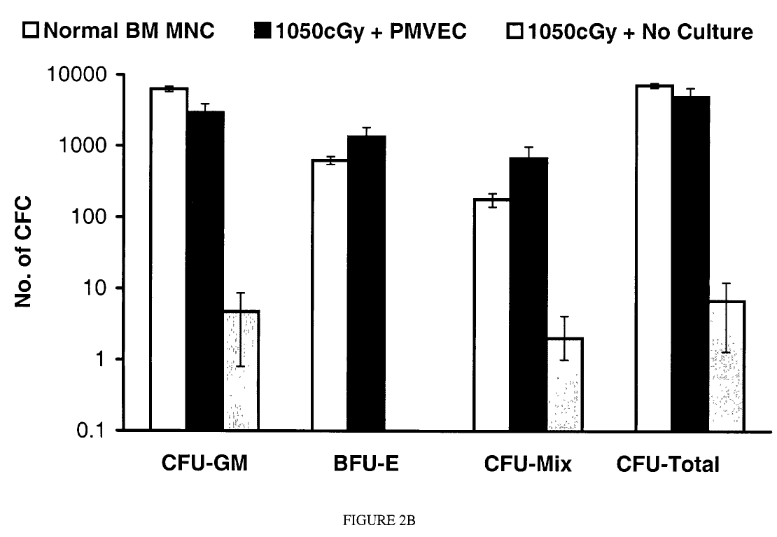

As shown in FIG. 2A, when BM MNC were harvested from irradiated C57B16 mice and subsequently placed in stroma-free liquid culture, we observed a 96% loss in cell numbers by day 3 and only 11% of the starting population remained by day 10. In comparison, BM MNC from irradiated mice which were cultured with PMVEC monolayers also decreased by 77% by day 3, but subsequently we observed an increase in viable cell numbers such that the cell count by day 10 recovered to 100% of the input number. The difference in recovery of irradiated hematopoietic cells following PMVEC co-culture (Day 10: 4.2×10⁵±0.8 cells; Input: 4×10⁵ BM MNC) compared to stroma-free culture (Day 10: 4.3×10⁴±0.5 cells; Input: 4×10⁵ BM MNC) was highly significant (p<0.0001; student's t test). Similarly, as measured by Trypan Blue exclusion, the percentage of viable cells evident in stroma-free liquid cultures at each time point was <50%, whereas the percentage of viable cells measured in PMVEC co-cultures was >90% throughout the culture period, suggesting differences in the recovery process in these two groups post-radiation. For comparison with the recovery of irradiated BM MNC, the ex-vivo expansion of normal BM MNC on PMVEC monolayers and in stroma-free liquid culture is shown in FIG. 2A.

Bone marrow MNC obtained from mice irradiated with 1050 cGy showed little or no colony forming capacity (cloning efficiency 0.0007%; FIG. 2B). In contrast, PMVEC co-culture of irradiated BM MNC supported the recovery of CFC with a cloning efficiency of 4.9% and the CFU-Total production approximated the CFC capacity of fresh Day 0 murine BM MNC (FIG. 2B). Stroma-free liquid culture did not support the recovery of any measurable CFC in 14 day methylcellulose cultures. The difference between the number of CFC measured in the PMVEC culture group as compared to the irradiated group or the liquid culture group was also highly significant (p=0.012; Wilcoxon rank sum test).

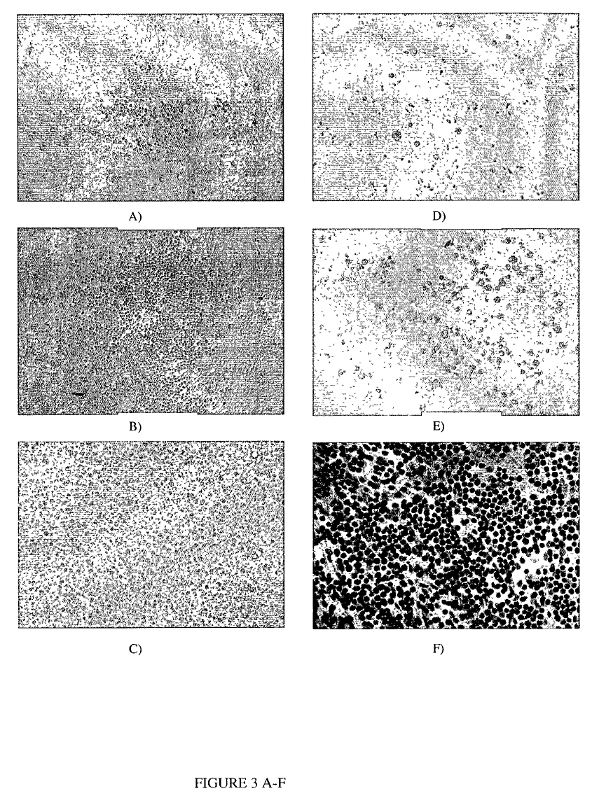

Using light microscopy, we were able to observe the recovery of irradiated BM progenitor cells on PMVEC monolayers over time. Seventy-two hours post radiation exposure, only a small population of adherent hematopoietic cells could be visualized on PMVEC monolayers and few intact cells were evident in stroma-free liquid cultures (FIGS. 3A, B). By day 7, cobblestone-like foci of hematopoietic cells had developed in PMVEC co-cultures (3C), whereas no recovery was evident in stroma-free liquid cultures (3D). At day 10, under higher magnification, sheets of hematopoietic cells were demonstrated in PMVEC co-culture (3E), and Wrights-Geimsa staining of the cells adherent to PMVEC monolayers showed a monomorphic population of cells with high nuclear:cytoplasmic ratio consistent with highly immature progenitor cells (3F).

Survival of Mice Transplanted with Irradiated BM Progenitor Cells

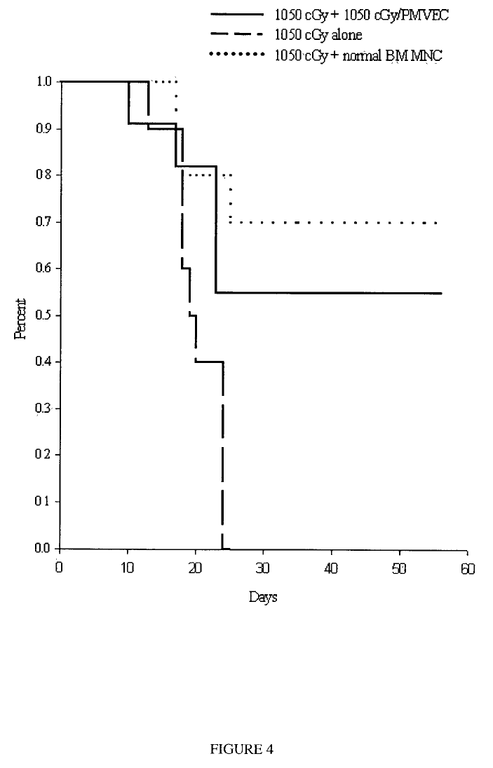

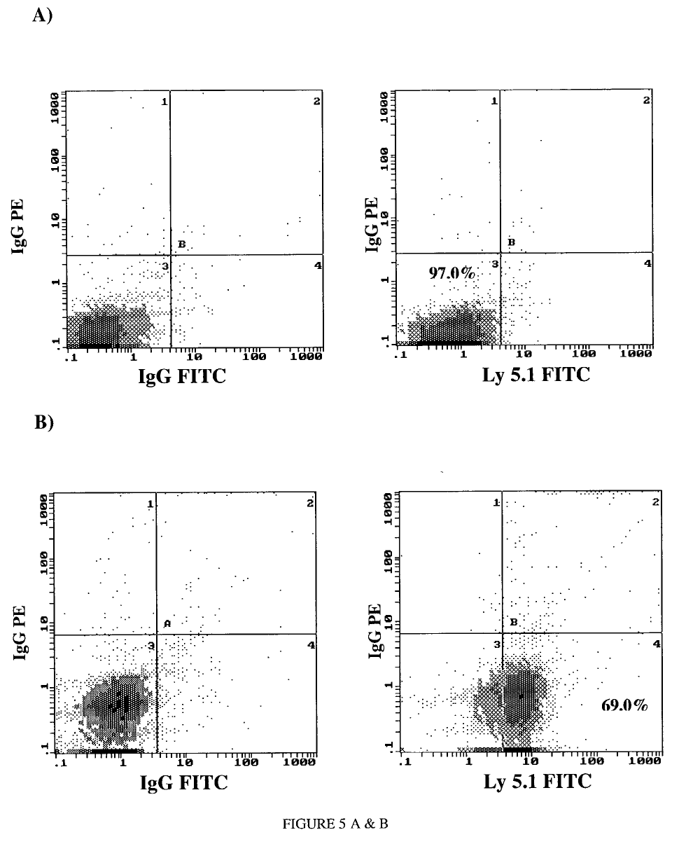

FIG. 4 shows the survival of Ly 5.2 recipient mice which were irradiated with 1050 cGy and transplanted with either irradiated BM MNC, irradiated/PMVEC-cultured cells, or normal donor BM MNC. Irradiated BM MNC which were transplanted at a dose of 2×10⁶ cells/graft were incapable of repopulating irradiated Ly 5.2 recipients (0 of 10 survival at day 30). In contrast, in 2 combined experiments, 6 of 11 (55%) irradiated Ly 5.2 recipients which were transplanted with 2×10⁶ irradiated/PMVEC-cultured cells remained alive and healthy at week 8. The survival duration of mice transplanted with irradiated/PMVEC-cultured cells was significantly greater than that of mice transplanted with irradiated BM MNC (p<0.01; student's t test). As a positive control, we also transplanted irradiated Ly 5.2 mice with 2×10⁶ normal BM MNC and 70% (7 of 10) of these animals remained alive and healthy after week 8. The percent survival of animals transplanted with irradiated/PMVEC-cultured cells was statistically no different than the survival of animals which received normal BM MNC (p>0.25).

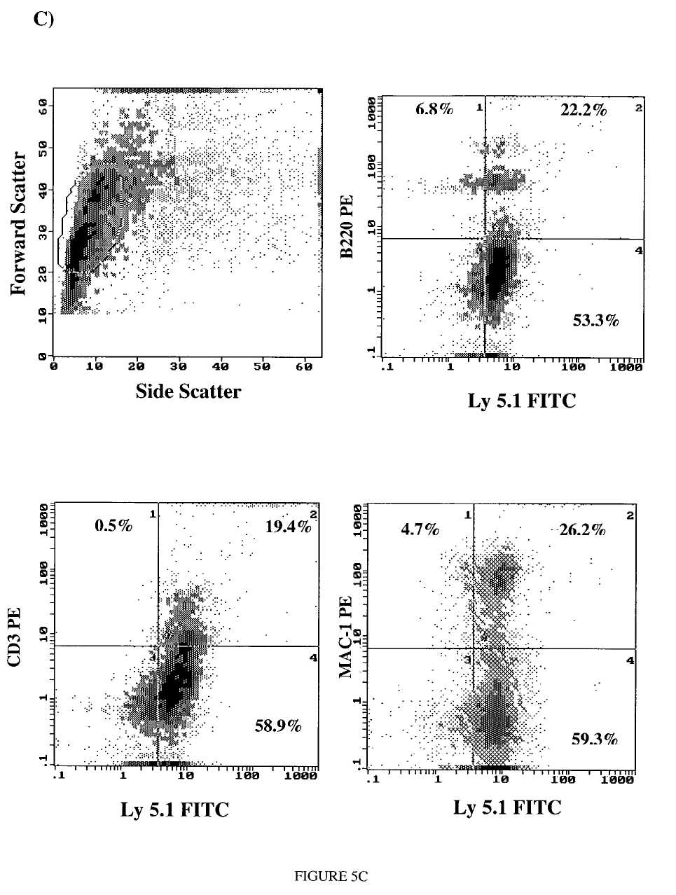

We also measured the percentage of donor Ly 5.1 cells which engrafted in the bone marrow of recipient Ly 5.2 mice at 8 weeks post transplant to compare this with the survival differences we observed. As shown in Table 1, Ly 5.2 mice transplanted with 1050 cGy irradiated/PMVEC Ly 5.1 cultured cells demonstrated a mean engraftment of 45.7% Ly 5.1⁺ cells in the bone marrow, and this level of engraftment was comparable to the mean engraftment (40.5% Ly 5.1⁺ cells) observed in recipient Ly 5.2 mice transplanted with normal donor Ly 5.1 BM MNC. Of note, >95% of the CD3⁺ T cell population in mice transplanted with irradiated/PMVEC cultured cells was derived from the donor cultured cell population at week 8 post-transplant. In contrast, animals transplanted with normal BM MNC showed <50% repopulation of the CD3⁺ subset with donor cells. In FIG. 5A, the lack of expression of Ly 5.1 within a representative normal female C57B16 mouse (Ly 5.2) is shown. In FIG. 5B, the expression of Ly 5.1 is shown in the marrow of a representative Ly 5.2 mouse which was transplanted with irradiated/PMVEC cultured Ly 5.1 cells. The tri-lineage distribution of Ly 5.1 donor cells in a Ly 5.2 recipient mouse transplanted with irradiated/PMVEC-cultured cells is shown in FIG. 5C. The initial scatter gating to exclude non-viable cells from this analysis is also shown in FIG. 5C. It will be appreciated to those skilled in the art that the invention can be performed within a wide range of equivalent methods? Without departing from the spirit or scope of the invention or any embodiment thereof.

TABLE 1
Engraftment of Irradiated/PMVEC-cultured cells vs. Normal
BM MNC vs. Irradiated BM MNC

| | | Per cent of Ly 5.1 donor | Per cent of engrafted Ly 5.1 | | |
|---|---|---|---|---|---|
| Group | | cells in bone marrow of | cells expressing: | | |
| (No. of animals) | Survival | recipient Ly 5.2 mice | B220 | Mac-1 | CD3 |
| Irradiated BM MNC + PMVEC | 6/11 | 50.6%, 58.0%, 22.2% | 30.8% ± 10.2 | 37.6% ± 7.2 | 24.2% ± 9.3 |
| Co-culture* | | 61.0%, 69.0%, 13.6% | | | |
| (n = 11) | | (mean 45.7% ± 22.5) | | | |
| Non-irradiated | 7/10 | 59.1%, 36.2%, 10.8% | 49.3% ± 14.3 | 25.5% ± 13.7 | 5.0% + 5.1 |
| Bone Marrow | | 53.4%, 58.8%, 63.1% | | | |
| MNC (n = 10) | | 2.3% (mean 40.5% ± 24.8) | | | |
| Irradiated BM MNC** | 0/10 | NM | NM | NM | NM |
| (n = 10) | | | | | |
| *Ly 5.1 donor mice were irradiated with 1050 cGy split dose (550/500 cGy) and, after 2 hours, the animals were sacrificed, bilateral femurs were removed and BM cells were collected via flushing with PBS at 4° C. The mononuclear cell fraction was collected using Ficoll-Hypaque centrifugation. Irradiated MNC were then plated in in co-culture with PMVEC monolayers at a starting dose of 4 × 10⁵ | | | | | |
| # cells/well for 10 days. At day 10, the non-adherent cells were collected, washed × 2 and then transplanted into irradiated (1050 cGy split) recipient Ly 5.2 mice at a dose of 2 × 10⁶ cells per animal. | | | | | |

**Ly 5.1 donor mice were irradiated with 1050 cGy split dose and their BM MNC were injected into recipient Ly 5.2 mice (dose: 2 × 10⁶ ells per animal).
NM = Engraftment percentages were not measurable as recipient mice died prior to week 8.

### References

1. Meijne E, Ria J, van der Winden-van Groenewegen R, Ploemacher R, et al. (1991) The effects of x-irradiation on hematopoietic stem cell compartments in the mouse. Exp Hematol 19:617.

2. McCarthy K (1997) Population size and radiosensitivity of murine hematopoietic endogenous long term repopulating cells. Blood 89:834.

3. Down J, Boudewijn A, van Os R, et al. (1995) Variations in radiation sensitivity and repair among different hematopoietic stem cell subsets following fractionated irradiation. Blood 86:122.

4. Inoue T, Hirabayashi Y, Mitsui H, et al. (1995) Survival of spleen colony-forming units (CFU-S) of irradiated bone marrow cells in mice: evidence for the existence of a radioresistant subfraction. Exp Hematol 23:1296

5. van Bekkum D. (1991) Radiation sensitivity of the hemopoieitc stem cell. Rad Res 128:S4

6. Baranov A, Gale R, Guskova A, et al. (1989) Bone marrow transplantation after the Chernobyl nuclear accident. N Engl J Med 32:205

7. Dainiak N, Sorba S (1997) Early identification of radiation accident victims for therapy of bone marrow failure. Stem Cells 15:275

8. Albanese J, Dainiak N (2000) Ionizing radiation alters fas ligand at the cell surface and on exfoliated plasma membrane-derived vesicles:implications for apoptosis and intercellular signaling. Rad Res 153:49

9. Belka C, Marini P, Budach W, et al. (1998) Radiation-induced apoptosis in human lymphocytes and lymphoma cells critically relies on the up-regularion of CD95/Fas/APO-1 ligand. Rad Res 149:588

10. Radford I, Murphy T (1994) Radiation response of mouse lymphoid and myeloid cell lines. Part III. Different signals can lead to apoptosis and may influence sensitivity to killing by DNA double-strand breakage. Int J Radiat Biol. 65:229

11. Chute J P, Saini A A, Kampen R L, Wells M R, Davis T A (1999) A comparative study of the cell cycle status and primitive cell adhesion molecule profile of human CD34+ cells cultured in stroma-free versus porcine microvascular endothelial cell cultures. Exp Hematol 27: 370.

12. Petzer et al. Petzer A L, Zandstra P W, Piret J M, Eaves C J (1996) Differential cytokine effects on primitive (CD34+CD38−) human hematopoietic cells: Novel responses to flt3-ligand and thrombopoietin. J Exp Med. 183: 2551

13. Galotto M, Berisso G, Delfino L, et al. (1999) Stromal damage as consequence of high dose chemo/radiotherapy in bone marrow transplant recipients. Exp Hematol 27:1460

14. Greenberger J S, Anderson J, Berry L, et al. (1996) Effects of irradiation of CBA/CA mice on hematopoietic stem cells and stromal cells in long term bone marrow cultures. Leukemia 10:514

15. Gorbunov N, Pogue-Geile K, Epperly M, et al. (2000) Activation of the nitric oxide synthase 2 pathway in the response of bone marrow stromal cells to high doses of ionizing radiation. Radiat Res 154:73

16. Hudak S, Leach M, Xu Y, Menon S, Rennick D (1998) Radioprotective effects of flk2/flt3 ligand. Exp Hematol 26:515

17. Neta R, Oppenheim J, Douches S (1987) Interdependence of the radioprotective effects of human recombinant interleukin 1 alpha, tumor necrosis factor, granulocyte colony stimulating factor, and murine recombinant granulocyte macrophage colony stimulating factor. J Immunol 140: 108

18. Zsebo K, Smith K, Hartley C, et al. (1992) Radioprotection of mice by recombinant stem cell factor. Proc Natl Acad Sci USA 89:9464

19. Davis T, Robinson D, Lee K, Kessler S (1995) Primary porcine microvascular endothelial cells support the in vitro expansion of human primitive hematopoietic bone marrow progenitor cells with a high replating potential: requirement for cell-to-cell interactions and colony stimulating factors. Blood 85:1751

20. Brandt J E, Galy A, Luens K, Travis M, et al. (1998) Bone marrow repopulation by human marrow stem cells following long term expansion culture on a porcine endothelial cell line. Exp Hematol 26:950

21. Brandt J E, Bartholemew A, Fortman J, Nelson M, et al. (1999) Ex vivo expansion of autologous bone marrow CD34+ cells with porcine microvascular endothelial cells results in a graft capable of rescuing lethally irradiated baboons. Blood 94: 106

22. Shalaby F, Rossant J, Yamaguchi T P, et al. (1995) Failure of blood island formation and vasculogenesis in flk-1 deficient mice. Nature 376:62

23. Hamaguchi I, Huang X L, Takakura N, et al. (1999) In vitro hematopoietic and endothelial cell development from cells expressing TEK receptor in murine aorta gonad mesonephros region. Blood 93:1549

24. Choi K, Kennedy M, Kazarov A, et al. (1998) A common precursor for hematopoietic and endothelial cells. Development 125:725

25. Zhao Y, Lin Y, Zhan Y, et al. (2000) Murine hematopoietic stem cell characterization and its regulation in BM transplantation. Blood 96:3016

26. Ploemacher R E, van Os R, van Beurden C A J, et al. (1992) Murine hemopoietic stem cells long term engraftment and marrow repopulating ability are more resistant to gamma-radiation than are spleen colony forming cells. Int J Radiat Biol 61:489

27. Eastgate J, Moreb J, Nick H, et al. (1993) A role for manganese superoxide dismutase in radioprotection of hematopoietic stem cells by interleukin-1. Blood 81:639

28. Ramakrishnan N, Wolfe W, Catravas G (1992) Radioprotection of hematopoietic tissues in mice by lipoic acid. Radiat Res 130:360

29. Tavassoli M (1982) Radiosensitivity of stromal cells responsible for in vitro maintenance of hemopoietic stem cells in continuous, long-term marrow culture. Exp Hematol 10: 435

30. Fried W, Chamberlin W, Kedo A, Barone J (1976) Effects of radiation on hematopoietic stroma. Exp Hematol 4:310

31. Gan O I, Murdoch B, Larochelle A, Dick J E (1997) Differential maintenance of primitive human SCID-repopulating cells, clonogenic progenitors, and long-term culture-initiating cells after incubation on human bone marrow stromal cells. Blood 90: 641

32. Thiemann F T, Moore K A, Smogorzewska E M, Lemischka I R, Crooks G M (1999) The murine stromal cell line AFT024 acts specifically on human CD34+CD38-progenitors to maintain primitive function and immunophenotype in vitro. Exp Hematol 26: 61

33. Morrison S J, Weissman I L (1994) The long term repopulating subset of hematopoietic stem cells is deterministic and isolatable by phenotype. Immunity 1:661

34. Peters S O, Kittler E L, Ramshaw H S, Quesenberry P J. (1995) Ex vivo expansion of murine marrow cells with interleukin-3 (IL-3), IL-6, IL-11, and stem cell factor leads to impaired engraftment in irradiated hosts. Blood. 87:30

35. Szilvassy S, Bass M, Van Zant G, Grimes B (1999) Organ selective homing define engraftment kinetics of murine hematopoietic stem cells and is compromised by ex-vivo expansion. Blood 93: 1557

36. Traycoff C M, Orazi A, Ladd A C, Rice S, et al. (1998) Proliferation-induced decline of primitive hematopoietic progenitor cell activity

We claim:

1. An ex-vivo method of treating myeloablation of hematopoietic stem cells and progenitor cells in a subject, comprising the steps of:
a. isolating said hematopoietic stem and progenitor cells from the bone marrow of a donor;
b. expanding said isolated stem and progenitor cells in a co-culture medium including endothelial cells;
c. harvesting said expanded stem and progenitor cells from said co-culture medium;
d. administering a therapeutic dose of said harvested expanded stem and progenitor cells to said myeloablated subject.

2. The method of claim 1, wherein the cause of said myeloablation in said subject is selected from the group consisting of: ionizing radiation, toxins, chemicals, drugs, disease, and genetic disorders.

3. The method of claim 2, wherein said hematopoietic cells are isolated from said donor or said subject within 24 hours after said myeloablation.

4. The method of claim 1, wherein said culture medium includes at least one cytokine.

5. The method of claim 4, wherein said at least one cytokine is selected from the group consisting of granulocyte-macrophage colony stimulating factor, interleukin-3, stem cell factor and interleukin-6, flt3-ligand, and mixtures thereof.

6. The method of claim 1, wherein said subject is a mammal.

7. The method of claim 1, wherein said medium comprises a monolayer of endothelial cells.

8. The method of claim 7, wherein said endothelial cells are porcine microvascular endothelial cells.

9. The method of claim 7, wherein said endothelial cells are human brain endothelial cells.

10. The method of claim 1, wherein said hematopoietic stem cells are CD34⁺38⁻.

11. The method of claim 1, wherein said hematopoietic stem cells are harvested after said expansion and before said cells differentiate.

12. The method of claim 11, wherein said harvesting occurs after 7 to 14 days of co-culture.

13. The method of claim 11, wherein said harvesting occurs after 10 to 14 days of co-culture.

14. The method of claim 1, wherein said culture media is maintained at a pH of about 7.2 to about 7.5 during said expanding step.

15. The method of claim 14, wherein said pH is maintained by replacing a portion of said culture medium with fresh culture medium.

16. The method of claim 1, wherein said therapeutic dose is about 1 to about 2 million cells/kg subject mass.

17. The method of claim 16, wherein said therapeutic dose is administered by injection.

18. The method of claim 17, wherein said therapeutic injection is administered by venous injection.

19. The method of claim 1, wherein said stem cells are CD34⁺.

20. The method of claim 1, wherein said method results in no engraftment defects in said co-cultured cells.

21. The method of claim 20, wherein said method results in no differentiation of the hematopoietic stem cell population.

22. The method of claim 21, wherein said method results in no alterations in adhesion molecules and homing receptors on said expanded hematopoietic stem cells.

23. The method of claim 1, wherein said donor and said subject are autologous.

24. The method of claim 1, wherein said donor and said subject are heterologous.

25. The method of claim 24, wherein said donor is myeloablated.

26. The method of claim 1, wherein said hematopoietic stem cells are enriched at least 85%.

27. The method of claim 1, wherein said hematopoietic stem cells are enriched at least 99%.

28. An ex-vivo method of restoring a depleted population of rapidly proliferating hematopoietic stem cells, comprising the steps of:
a. isolating hematopoietic stem cells from a donor;
b. expanding said isolated hematopoietic stem cells in a co-culture medium including endothelial cells;
c. harvesting said expanded hematopoietic stem cells from said co-culture medium;
d. administering a therapeutic dose of said harvested expanded hematopoietic stem cells to a subject.
